(19) United States
(12) Patent Application Publication
(10) Pub. No.: US 2013/0065957 A1
(43) Pub. Date: Mar. 14, 2013

(54) DICLOFENAC SALT OF TRAMADOL

(71) Applicants: Yung Shin Pharm. Ind. Co., Ltd., Tachia (TW); Carlsbad Technology, Inc., Carlsbad, CA (US)

(72) Inventors: Chia-Hui CHEN, Miaoli (TW); Yu-Liang HUANG, Taichung (TW); Liang-Rern KUNG, Hsinchu (TW); Ming-Chung YAN, Taichung (TW)

(73) Assignees: CARLSBAD TECHNOLOGY, INC., Carlsbad, CA (US); YUNG SHIN PHARM. IND. CO., LTD., Tachia (TW)

(21) Appl. No.: 13/666,786

(22) Filed: Nov. 1, 2012

Related U.S. Application Data
(63) Continuation of application No. PCT/US2011/043528, filed on Jul. 11, 2011.
(60) Provisional application No. 61/363,344, filed on Jul. 12, 2010.

(51) Int. Cl. A61K 31/196; C07C 227/18; A61P 25/00; C07C 229/42

(52) U.S. Cl. 514/555; 562/456

(57) ABSTRACT

The present invention relates to a compound of diclofenac-tramadol salt in 1:1 ratio and a pharmaceutical formulation comprising such compound. The present invention also relates to a method for treating a patient with moderate to moderately severe pain. The method comprises: identifying a patient suffering from moderate to moderately severe pain with pain intensity scale of 5-9, and administering to said patient the diclofenac-tramadol salt, in an effective amount. The method is particularly useful in treating postoperative pain after Cesarean, postoperative pain after non-Cesarean surgeries, cancer pain, osteoarthritis pain, or rheumatoid arthritis pain.

This application is a continuation of PCT/US2011/043528, filed Jul. 11, 2011; which claims the priority of U.S. Provisional Application Nos. 61/363,344, filed Jul. 12, 2010. The contents of the above-identified applications are incorporated herein by reference in their entireties.

### TECHNICAL FIELD

This application relates to a compound of diclofenac-tramadol salt in 1:1 ratio. This application also relates to the use of the compound in treating moderate to moderately severe pain such as postoperative pain (e.g., after cesarean delivery or other surgeries), cancer pain, and pain associated with erosive osteoarthritis and rheumatoid arthritis.

### BACKGROUND OF THE INVENTION

Based on the physical causes, pain can be divided into three types: nociceptive, neuropathic, and mix-type.

Nociceptive pain is usually caused by noxious stimulation such as heat and cut that directly results in damage or injury to the body or tissue. Based on the initiation site of the pain, nociceptive pain can be further divided into two types: somatic and visceral pain. Somatic pain arises from bone, joint, muscle, skin, or connective tissues that directly in contact with the external noxious stimuli. Visceral pain arises from compression, extension, and injury of the internal organs. Most people describe the symptoms as achy, sharp, stinging, and throbbing. Nociceptive pain is usually short in duration and end when the damage recovers. Examples of nociceptive pain include postoperative pain, sprains, bone fractures, burns, bumps, bruises, and inflammatory pain (with the exception of inflammation caused by arthritis).

Neuropathic pain is originated from spontaneous ectopic neuron discharge in the nervous system either in central or in peripheral. Due to the underlying etiologies are usually irreversible, most of neuropathic pain are chronic pain. Most people describe neuropathic pain as shooting, burning, tingling, lancinating, electric shock qualities, numbness, and persistent allodynia. The nomenclature of neuropathic pain is based on the site of initiating nervous system with the etiology; for examples, central post-stroke pain, diabetes peripheral neuropathy, post-herpetic (or post-shingles) neuralgia, terminal cancer pain, phantom limb pain.

Mix-type pain is featured by the coexistence of both nociceptive and neuropathic pain. For example, muscle pain trigger central or peripheral neuron sensitization leading to chronic low back pain, migraine, and myofacial pain.

Clinically pain intensity is rated on a scale of 0 to 10; with 0 is no pain, 1-3 is mild pain, 4-6 is moderate pain, and 7-10 is severe pain. For example, 8-9 is designated for moderately severe pain.

WHO “3-Step” Guideline provides the guideline for managing pain. The “3-Step” is determined by the pain intensity and the analgesia activity of drugs.

(a) 1st Step mild pain: Acetaminophen, NSAIDs, or combination of both. Common used NSAIDs including aspirin, diclofenac, indomethacin, sulindac, ketoprofen, etodolac, ketorolac.

(b) 2nd Step moderate pain: NSAIDs+opiate, including aspirin or acetaminophen with codein, oxycodon, dihydrocodein, hydrocodon, tramadol

(c) 3rd Step severe pain: Strong opiate including morphine, hydromorphine, methadol, levorphanol, fentanyl, oxycodon

It is well recognized that acetaminophen, NSAIDs, and opioids all have their inherent drawbacks. Acetaminophen and NSAIDs often exhibit ceiling effect (upper limit of pain relief). Once that upper limit is reached, taking additional medication provides no further pain relief. In addition, NSAIDs has end organ toxicities in heart, liver, GI tract, and kidney at the regular doses. Opioids usually cause intolerable adverse effects such as constipation, respiratory depression, physical dependence and abuse problems. Primarily, NSAIDs provide peripheral anti-nociception and opioids provide central anti-nociception.

Ketorolac tromethamine is a potent nonsteroidal anti-inflammatory drug (NSAID) with analgesic efficacy similar to opioids. This drug is administered to treat moderate pain or, combined with reduced opioid doses, for severe pain.

Tramadol (molecular weight 263.4), (1R,2R)-rel-2-[(dimethylamino)methyl]-1-(3-methoxyphenyl)cyclohexanol, is in a class of opiate agonists. Tramadol is classified as a central nervous system drug usually marketed as the hydrochloride salt (tramadol hydrochloride). Tramadol hydrochloride is a centrally acting opioid analgesic, used in treating moderate to severe pain. The drug has a wide range of applications, including treatment for restless leg syndrome and fibromyalgia.

### SUMMARY OF THE INVENTION

The present invention is directed to a compound of diclofenac-tramadol salt in 1:1 ratio and a pharmaceutical formulation comprising such compound. In one embodiment, the compound is in a crystalline form.

The diclofenac-tramadol salt (1:1) can be prepared by mixing diclofenac and tramadol in a solvent or solvent mixture, followed by removing the solvent or solvent mixture.

The present invention is also directed to a method for treating a patient with moderate to moderately severe pain. The method comprises: identifying a patient suffering from moderate to moderately severe pain with pain intensity scale of 5-9, and administering to said patient the diclofenac-tramadol salt (1:1), in an effective amount. The method is particularly useful in treating postoperative pain after Cesarean, postoperative pain after non-Cesarean surgeries, cancer pain, osteoarthritis pain, or rheumatoid arthritis pain.

### BRIEF DESCRIPTION OF THE DRAWINGS

FIG. 1 shows a thermogram of differential scanning calorimetry (DSC) spectrum of the diclofenac salt of tramadol obtained from Example 1.

FIG. 2 shows the profile of weight loss versus temperature curve in thermo-gravimetric analysis (TGA) spectrum of the diclofenac salt of tramadol obtained from Example 1.

FIG. 3 shows the single crystal X-Ray diffraction data analysis of a single crystal of the diclofenac salt of tramadol obtained from Example 1.

### DETAILED DESCRIPTION OF THE INVENTION

Diclofenac is a non-steroidal anti-inflammatory drug (NSAID) and acts as an analgesic in certain conditions. It is a weak acid.

Tramadol is in a class of analgesics called opiate agonists. It is a weak base.

The present invention provides a novel compound of diclofenac-tramadol salt in 1:1 ratio, which exhibits combined therapeutic effects of diclofenac acid and tramadol. Diclofenac-tramadol salt and diclofenac salt of tramadol is used interchangeable in this application. Diclofenac-tramadol salt (molecular weight 559.55), which is an NSAID salt of an opiate agonist, is characterized by its distinctive physical and chemical properties, which are different from either diclofenac alone or tramadol alone, as demonstrated by the DSC, TGA, HPLC, and FTIR analyses.

The diclofenac-tramadol salt is composed of two types of analgesics, diclofenac and tramadol. The resulting peripheral and central analgesia activities make diclofenac salt of tramadol a “balanced analgesic,” which can be used in a wider spectrum of the pain management with much reduced unwanted side effects. Diclofenac salt of tramadol acts from peripheral to reduce nociceptive input and from central to enhance natural anti-nociceptive response.

The diclofenac-tramadol salt provides combined benefits of the two individual drugs of diclofenac and tramadol. The advantages of diclofenac-tramadol salt include: (a) providing shorter analgesia onset and prolonged duration, (b) providing both central and peripheral analgesic effect by complementary mechanisms of actions, (c) reducing the dose of each drug and thus minimizing side effects, (d) improving water solubility in comparison with its parent drugs. Further, since NSAIDs can only manage mild to moderate pain due to a ceiling effect, diclofenac salt of tramadol is superior to diclofenac alone, because the addition of tramadol eliminates the ceiling effect of diclofenac. Thus, diclofenac salt of tramadol can be used to treat pain with moderately severe intensity. These improvements offer more treatment options to patients.

The diclofenac-tramadol salt (1:1 ratio) can be an amorphous form or a crystalline form. In one embodiment, the crystalline form of the diclofenac-tramadol salt is characterized by a powder X-ray diffraction pattern, having X-ray diffraction peaks at about 11.0°, 19.0°, 20.5° and 20.8°±0.2° 2θ.

Tramadol is a basic compound and it is capable of forming pharmaceutically acceptable acid addition salts with strong or moderately strong, non-toxic, organic or inorganic acids by methods known to the art. Exemplary of the acid addition salts are maleate, fumarate, lactate, oxalate, methanesulfonate, ethanesulfonate, benzenesulfonate, tartrate, citrate, hydrochloride, hydrobromide, sulfate, phosphate and nitrate salts.

Diclofenac acid is an acidic compound and is capable of forming acceptable base addition salts with organic and inorganic bases by conventional methods. Examples of the nontoxic alkali metal and alkaline earth bases include, but are not limited to, calcium, sodium, potassium and ammonium hydroxide; and nontoxic organic bases include, but are not limited to, triethylamine, butylamine, piperazine, and tri(hydroxymethyl)-methylamine.

### Method of Preparing Diclofenac-Tramadol Salt

The diclofenac-tramadol salt (1:1) can be prepared by mixing diclofenac and tramadol base in a solvent or solvent mixture, followed by removing the solvent or solvent mixture (see scheme 1 below). Preferably, about equal molars of diclofenac acid and tramadol base are mixed.

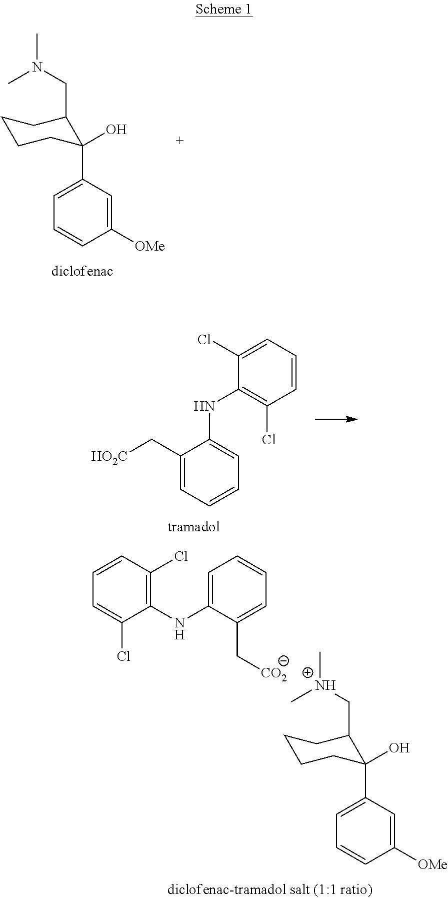

In one embodiment, the method comprises: (a) dissolving a diclofenac in a first solvent to form a first solution, (b) dissolving tramadol in a second solvent to form a second solution, (c) mixing the first solution and the second solution to form a mixture, and (d) removing the first solvent and the second solvent from the mixture to form the pharmaceutically compound. The first solvent and the second solvent are selected from the group consisting of dichloromethane, ethyl acetate, methanol, ethanol, isopropyl alcohol, acetone, toluene, chloroform, dimethylformamide, dimethylacetamide, dimethylsulfoxide, methylene chloride, and acetonitrile. Diclofenac is preferably dissolved in dichloromethane. Tramadol is preferably dissolved in ethyl acetate.

Alternatively, the diclofenac-tramadol salt (1:1) can be prepared by a method comprising (a) dissolving a diclofenac and tramadol in a solvent or a solvent mixture, and (b) removing the solvent or the solvent mixture to form the pharmaceutically compound. The solvent and the solvent mixture are selected from the group consisting of dichloromethane, ethyl acetate, methanol, ethanol, isopropyl alcohol, acetone, toluene, chloroform, dimethylformamide, dimethylacetamide, dimethylsulfoxide, methylene chloride, acetonitrile, and a combination thereof.

The solvent or solvent mixture of the above method can be removed by evaporation, vacuum condensation, or drying under nitrogen.

The diclofenac salt of tramadol can be filtered and dried, and, optionally, can be re-purified by re-dissolving the salt in a suitable solvent followed by drying to remove the solvent.

### Characterization of Diclofenac Salt of Tramadol

Diclofenac salt of tramadol (1:1 ratio) can be characterized by the following analysis. The results are described in Example 2.

Thermal Analysis: Two thermal analysis, thermogravimetric analysis (TGA) and differential scanning calorimetry (DSC) can be used. TGA measures the change in the mass of sample as the temperature is changed. The profile of the overall thermogravimetric weight loss versus temperature curve provides reliable indication of the phase and weight changes of the pharmaceutical compounds.

DSC examines the changes in physical properties of the pharmaceutical compound with temperature or time. During operation, DSC heats the test sample, measures heat flow between the test sample and its surrounding environment, and records a test thermogram of the test sample based on the measured heat flow. DSC provides information regarding the onset temperature, the endothermal maximum of melting, and the enthalpy of the pharmaceutical compound.

High Performance Liquid Chromatography (HPLC): The content and/or purity of the diclofenac-tramadol salt can be determined by HPLC method.

UV Spectroscopy: The UV spectroscopy can be used to perform qualitative analysis of the diclofenac-tramadol salt.

Infrared (IR) Spectroscopy including Fourier-Transformed Infrared Spectroscopy (FTIR): Functional groups of the diclofenac-tramadol salt can be determined by IR spectra based on their respective light transmittance. An FTIR microscope allows the measurement of the IR spectrum of a single crystal or a group of crystals.

Liquid Chromatography-Mass Spectroscopy (LC-MS): The molecular weight and the chemical structure of the diclofenac-tramadol salt can be determined using the liquid chromatography-mass spectroscopy (LC-MS) method.

### Pharmaceutical Formulations

The present invention is directed to a pharmaceutical formulation comprising diclofenac-tramadol salt (1:1 ratio) and a pharmaceutically acceptable carrier. The pharmaceutical acceptable carriers are in general those commonly used and generally recognized by person skilled in the art of pharmaceutical formulation.

The pharmaceutical formulations of the present invention are particularly suitable for injection, topical application, and oral administration. In the injection solution, the diclofanac salt of tramadol is, for example, first dissolved in benzyl alcohol, then mixed with methyl paraben and propyl paraben, before the addition of water.

Formulations suitable for topical administration include liquid or semi-liquid preparations suitable for penetration through the skin to the site of where treatment is required. Examples of liquid preparations include, but are not limited to, topical solutions or drops. Examples of semi-liquid preparations include, but are not limited to, liniments, lotions, creams, ointments, pastes, gels, emugels. Topical solution or drops of the present invention may contain aqueous or oily solution or suspensions. They may be prepared by dissolving the diclofenac-tramadol salt in a suitable aqueous solution of a bactericidal and/or fungicidal agent and/or any other suitable preservative, and optionally including a surface active agent.

Examples of bactericidal and fungicidal agents suitable for inclusion in the solution include, but are not limited to, phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol. Optionally, L-menthol can be added to the topical solution.

Lotions and liniments include those suitable for application to the skin, which may contain a sterile aqueous solution and optionally a bactericide. They may also include an agent to hasten drying and cooling of the skin, such as alcohol or acetone, and/or a moisturizer such as glycerol or an oil such as castor oil or arachis oil.

Cream, ointments or pastes are semi-solid formulations. They may be made by mixing the pharmaceutically acceptable salts in finely-divided or powdered form, alone or in solution or suspension in an aqueous or non-aqueous fluid, with the aid of suitable machinery, with a greasy or non-greasy base. The base may contain hydrocarbons. Examples of the hydrocarbons include, but are not limited to, hard, soft, or liquid paraffin, glycerol, beeswax, a metallic soap, a mucilage, an oil of natural origin (such as almond, corn, arachis, castor or olive oil), wool fat or its derivative, and/or a fatty acid (such as stearic acid or oleic acid). The formulation may also contain a surface active agent, such as anionic, cationic or non-ionic surfactant. Examples of the surfactants include, but are not limited to, sorbitan esters or polyoxyethylene derivatives thereof (such as polyoxyethylene fatty acid esters), and carboxypolymethylene derivatives thereof (such as carbopol). Suspending agents such as natural gums, cellulose derivatives inorganic materials such as silicaceous silicas, and other ingredients such as lanolin, may also be included. For ointments, polyethylene glycol 540, polyethylene glycol 3350, and propyl glycol may also be used to mixed with the pharmaceutical compound.

A gel or emugel formulation of the present invention includes any gel forming agent commonly used in pharmaceutical gel formulations. Examples of gel forming agents are cellulose derivatives such as methyl cellulose, hydroxyethyl cellulose, and carboxymethyl cellulose; vinyl polymers such as polyvinyl alcohols, polyvinyl pyrrolidones; and carboxypoly-methylene derivatives such as carbopol. Further gelling agents that can be used for the present invention are pectins, gums (such as gum arabic and tragacanth, alginates, carrageenates, agar and gelatin). The preferred gelling agent is carbopol. Furthermore, the gel or emugel formulation may contain auxiliary agents such as preservatives, antioxidants, stabilizers, colorants and perfumes.

### Pharmaceutical Use of the Diclofenac-Tramadol Salt

Based on the known analgesic-related mechanism of actions and reported clinical potentials, the diclofenac salt of tramadol of the present invention is useful in the management of pain of moderate to moderately severe intensity (scale 5-9), preferably in the management of pain of moderately severe intensity (scale 8-9). The diclofenac salt of tramadol is particularly useful in the management of postoperative pain, cancer pain, erosive osteoarthritis, and rheumatoid arthritis.

The present invention is also directed to a method for treating a patient with moderate to moderately severe pain. The method comprises: identifying a patient suffering from moderate to moderately severe pain with pain intensity scale of 5-9, and administering to said patient the diclofenac-tramadol Salt, in an effective amount. “An effective amount,” as used herein, refers to an amount that is effective to reduce or relief pain from a patient.

The present method is particularly useful in treating moderately severe pain with pain intensity of 8-9. The method is also particularly useful in treating postoperative pain after Cesarean, postoperative pain after other surgeries, severe cancer pain, osteoarthritis pain, or rheumatoid arthritis pain. In the above treatments, an effective amount of the diclofenac-tramadol salt is in general about 50-200 mg/dose, or 65-175 mg/dose, or 100-150 mg/dose. A preferred dosage is about 125-135 mg/dose, e.g., about 131 mg/dose (equivalent to 75 mg diclofenac sodium and 70 mg tramadol.HCl). “About” as used herein, refers to ±10% of the recited value.

### Postoperative Pains

Postoperative pain is resulted from the somatic pain and visceral pain. The somatic pain arises from the direct noxious impulse at injury sites (cut). The sensitization of afferent fibers at injury sites driving central sensitization. Visceral pain arises from the compression, extension, or inflammation of internal organs. Diclofenac salt of tramadol offers benefit to patients whose pain is caused from inflammation (COX1/COX2) and visceral pain. It also enhances antinociceptive response from central by modulation of μ receptor and the level of serotonin and noradrenalin

Diclofenac salt of tramadol is particularly effective in the management of postoperative pain after cesarean for the following reasons:

(a) Visceral pain caused by uterus contraction is the main component of pain after cesarean delivery. Diclofenac is highly effective in relief visceral pain.

(b) Prostaglandin involves in both tissue injury and uterus contraction. Inhibition of PGE2 by diclofenac offer better anti-inflammatory and analgesic effects than acetaminophen alone.

(c) Diclofenac does not induce uterine atony, which is the main reason of postpartum hemorrhage. Diclofenac is therefore safer than other NSAIDs in the control of Cesarean pain.

(d) The mechanisms of action of diclofenac salt of tramadol responsible for Cesarean pain relief including inhibition on COX1 and COX2, sodium current, serotonin and noradrenaline reuptake and activation of g receptor.

Over half of the postoperative pain patients still experience inadequate pain relief with currently available treatment. Diclofenac salt of tramadol is useful in the pain control after other types of surgery, such as coronary artery bypass grafting (CABG), lumbar disc surgery, orthopaedia, and tonsillectomy.

Postoperative pain is mostly acute and severe. The management of postoperative pain usually starts at one hour after surgery and continue for another 48-72 hours. Since most patients are hospitalized after major surgeries, parenteral administration of analgesics is considered to be easy and convenient. For diclofenac salt of tramadol, which are subjected to the first-pass effect via significant metabolism in GI tract and liver following oral administration, parenteral administration offers additional benefit in the increase of drug exposure and shorter onset. In addition, drug absorption can be variable in the first 24 hours following surgery. The preferred route of administration for post operative pain is intramuscular (IM) injection.

### Cancer Pain

Cancer pain is the result of tissue damage caused by the tumor, the effects of chemotherapy, radiation, or surgery. Cancer pain can occur at any stage of cancer. The pain intensity ranges from moderate to severe pain. Cancer patients at terminal phase often experience an intolerable severe pain, in which the highly potent opioids like morphine are commonly used.

Prostaglandins-induced inflammation and nociceptor sensitization contributes to a varying extent in the process of cancer and exacerbation of nociception. Cancer pain can be nociceptive, neuropathic, or both depending on the course of cancer. Diclofenac salt of tramadol offers benefit to the cancer patients whose pain results from severe inflammation (prostaglandin) and abnormal excitability of sodium channels. It also enhances anti-nociceptive response from central by modulation of μ receptor and the level of serotonin and noradrenalin.

Diclofenac salt of tramadol is useful in the management of cancer pain for the following reasons:

(a) Cancer pain is mainly both inflammatory and neuropathic. Diclofenac salt of tramadol, which offers both anti-inflammation and nerve block activity, is effective in the management of cancer pain with moderately severe intensity.

(b) Diclofenac salt of tramadol can be used in cancer patients at terminal stage for reducing the use of morphine in pain relief.

Terminal stage cancer patients with severe pain can be acute and chronic with occasional breakthrough pain with moderate to severe intensity. For the management of acute and breakthrough pain, parenteral administration by IM or IV provides rapid and effective pain relief. For the management of chronic pain, oral or topical administration of diclofenac salt of tramadol is preferred. While some cancer patients are suffering from swallowing problems, sublingual or oral disintegration tablet (ODT) is a good choice for oral administration.

### Osteoarthritis and Rheumatoid Arthritis

Osteoarthritis (OA) is a degenerated disorder attributed to loss of resilient in joints due to the reduction of proteoglycan in cartilage. Primary osteoarthritis has two subtypes; nodal osteoarthritis is resulted from the reduced proteoglycan in cartilage, and erosive osteoarthritis where inflammation is also present in the surrounding joint capsule in addition to the reduced proteoglycan in cartilage. Pain associated with osteoarthritis arises from inflammation and possibly peripheral neuropathy when the inflammation results in the damage or dysfunction of peripheral nerves.

Rheumatoid arthritis (RA) is a chronic inflammatory disorder. Increase of prostaglandin has been found in affected tissues. Autoimmune also play roles in the progression of rheumatoid arthritis. Pain associated with rheumatoid arthritis mainly results from severe inflammation.

Diclofenac salt of tramadol is useful in treating rheumatoid arthritis pain and osteoarthritis pain, particularly erosive osteoarthritis pain, because pain from inflammation responds better to antiinflammatory drugs.

Pain associated with erosive osteoarthritis and rheumatoid arthritis is generally chronic with moderate to severe intensity. Oral (e.g. controlled-release tablet) or topical (e.g., topical gel) administration of diclofenac salt of tramadol is a convenient and cost-effective treatment.

The invention is illustrated further by the following examples that are not to be construed as limiting the invention in scope to the specific procedures described in them.

### EXAMPLES

### Example 1

### Process to Obtain Tramadol-Diclofenac (1:1) Co-Crystal

A solution of tramadol free base (22 g, 83.5 mmol) in 50 mL dichloromethane was added to a stirred solution of diclofenac free acid (24.7 g, 83.4 mmol) in 50 mL ethyl acetate. The mixture was heated until dissolution and the solvent was evaporated under vacuum to produce white amorphous solid. The amorphous solid was dissolved in 350 mL acetonitrile and heated to 75° C. with stiffing for 1 h. The clear solution was cooled to room temperature to generate white precipitation. The obtained precipitation was filtered off and dried by oven to give diclofenac salt of tramadol in a 1:1 ratio as a crystalline white solid (43.6 g, 93.2% yield). This crystalline form of the diclofenac salt of tramadol, characterized by a powder XRD pattern, having X-ray powder diffraction peaks selected from the following: at about 11.0°, 19.0°, 20.5° and 20.8°±0.2° 2 θ. In the NMR study of this diclofenac salt of tramadol and tramadol free base, the large shift (about 0.2 ppm) of the peak of the two methyl groups of tramadol's tertiary amine proved that the tertiary amine of tramadol was protonated in diclofenac salt of tramadol.

### Example 2

### Identification of Diclofenac Salt of Tramadol (1:1)

The diclofenac salt of tramadol (1:1 ratio) of Example 1 is identified by the following analyses.

### NMR

Proton nuclear magnetic resonance (NMR) analyses of sample from Example 1 were recorded in deuterated chloroform (CDCl₃) in a Bruker Avance II 400 Ultrashield NMR spectrometer.

Diclofenac salt of tramadol: ¹H NMR (400 MHz, CDCl₃) δ 8.12 (br, 1H, proton of protonated tertiary amine), 7.31 (d, J=8.0 Hz, 1H), 7.25 (t, J=8.0 Hz, 1H), 7.22 (dd, J=6.0, 1.6 Hz, 1H), 7.09 (br, 1H), 7.03 (td, J=7.6, 1.6 Hz, 1H), 6.98 (d, J=8.0 Hz, 1H), 6.93 (t, J=8.0 Hz, 1H), 6.86 (td, J=7.6, 1.2 Hz, 1H), 6.75 (dd, J=8.0, 2.4 Hz, 1H), 6.49 (dd, J=7.6, 1.2 Hz, 1H) 3.80 (s, 3H), 3.75 (s, 2H), 2.72 (dd, J=13.2, 8.0 Hz, 1H), 2.29 (s, 6H), 2.25 (dd, J=13.2, 2.0 Hz, 1H), 1.94-2.00 (m, 1H), 1.82-1.86 (m, 1H), 1.52-1.79 (m, 6H), 1.24-1.31 (m, 1H) ¹³C NMR (100 MHz, CDCl₃) δ 177.64, 159.63, 149.65, 142.88, 138.33, 130.70, 129.46, 129.31, 128.78, 126.77, 126.66, 123.31, 121.15, 117.22, 117.10, 111.61, 111.03, 75.64, 60.35, 55.19, 44.82, 42.75, 41.59, 41.04, 26.88, 25.50, 21.63.

### Differential Scanning Calorimetry (DSC) Spectrum

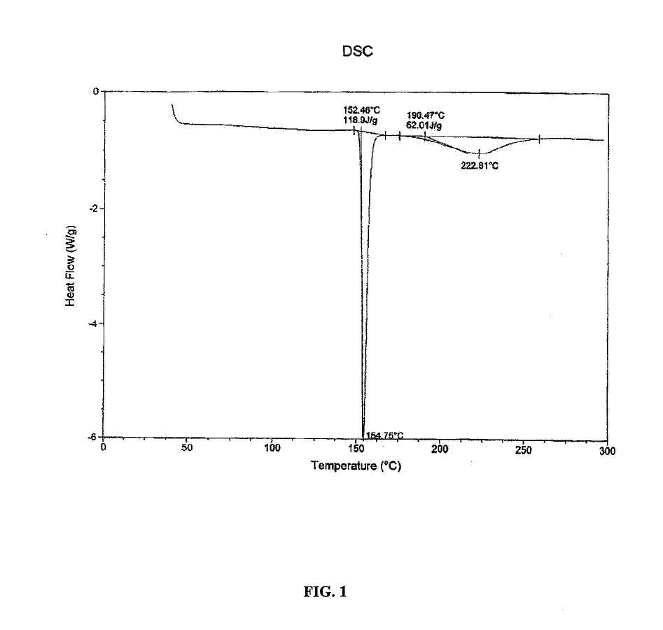

FIG. 1 shows a thermogram of differential scanning calorimetry (DSC) spectrum of the amorphous cabazitaxel. The DSC was run at a heating rate of 10° C./min. There was one endothermal bands in the spectrum. The onset temperature was at 143.62° C. and the endothermal maximum of melting is at 145.06° C.

### Thermo-Gravimetric Analysis (TGA) Spectrum

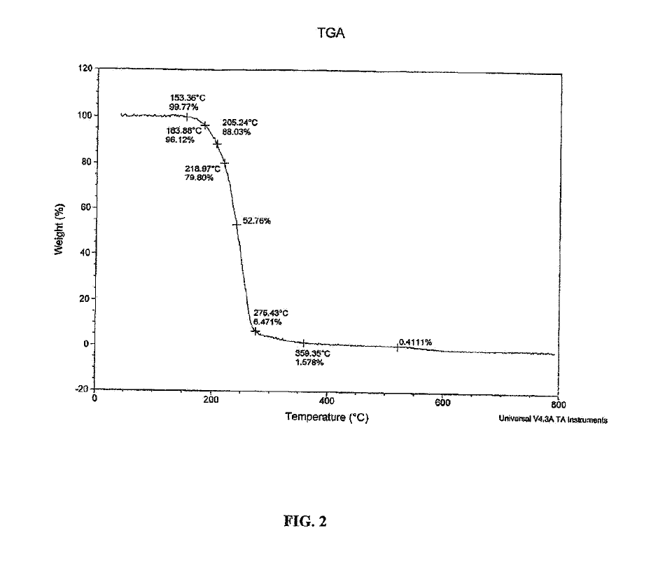

FIG. 2 shows the profile of weight loss versus temperature curve in thermo-gravimetric analysis (TGA) spectrum of the diclofenac salt of tramadol obtained from Example 1. The TGA was run at a heating rate of 10° C./min. Shown in the curve is the % of weight remained of the salt at 153.36° C., 183.88° C., 205.24° C., 218.97° C., 275.43° C., and 359.35° C. At 275.43° C., the % of weight remained was 6.47%.

### FTIR Spectrum

The FTIR spectrum (ATR) of the co-crystal of tramadol-diclofenac (1:1) of Example 1 was recorded using a PerKin Elmer Spectrum 100. The FTIR spectrum was shown with absorption bands νₘₐₓ at 3346, 3078, 2945, 2845, 2362, 1604, 1587, 1500, 1373, 1177, 1152, 1101, 1046, 1011, 985, 960, 927, 891, 836, 775, 755, 717, 704, and 646 cm⁻¹. The peaks show the special functional groups of tramadol and diclofenac.

### Single Crystal X-Ray Diffraction

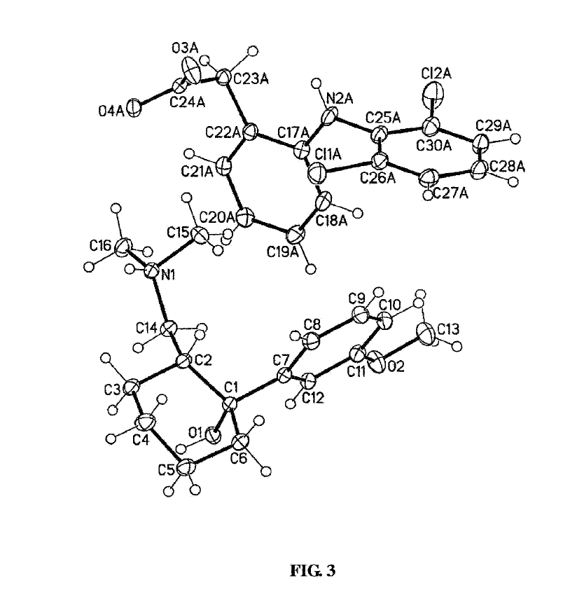

The crystal structure of tramadol-diclofenac (1:1) of Example 1 was determined from single crystal X-ray diffraction data (SCXRD). The crystal structure determination of the sample obtained from Example 1 was carried out using a Bruker-SMART APEX diffractometer. FIG. 3 shows the single crystal X-Ray diffraction data analysis of a single crystal of the diclofenac salt of tramadol obtained from Example 1. All non hydrogen atoms were refined including anisotropic displacement parameters. Crystal data and structure refinement for a co-crystal of tramadol-diclofenac (1:1) is given in Table 1.

TABLE 1

| Crystal system | Monoclinic |
|---|---|
| Space group | P-1 |
| A(Å) | 7.7589(4) |
| B(Å) | 10.3773(5) |
| C(Å) | 17.5868(9) |
| α | 100.9540(10) |
| β | 93.1720(10) |
| γ | 93.8130(10) |
| Z | 2 |
| Volume(Å ³) | 1383.81(12) |

### Powder X-Ray Diffraction Analysis

Powder X-ray diffraction (PXRD) analysis of the sample obtained from Example 1 was performed using a SHIMADZU XRD-6000 diffractometer. The measurement parameters were as follows: the range of 2θ was 10° to 40° at a scan rate of 2° per minute. The peaks expressed in angles 2θ and d-values are described in detail in Table 2.

TABLE 2

| Angel | d-Valued | Relative | Angel | d-Valued | Relative |
|---|---|---|---|---|---|
| 2θ¹ | (Å) | Intensity % | 2θ¹ | (Å) | Intensity % |
| 10.34 | 8.55 | 29 | 21.92 | 4.05 | 32 |
| 10.80 | 8.19 | 13 | 22.98 | 3.87 | 8 |
| 10.99 | 8.04 | 61 | 23.34 | 3.81 | 17 |
| 12.33 | 7.17 | 27 | 24.12 | 3.69 | 27 |
| 13.64 | 6.49 | 33 | 26.22 | 3.40 | 6 |
| 14.08 | 6.29 | 18 | 27.24 | 3.27 | 5 |
| 14.33 | 6.18 | 18 | 28.01 | 3.18 | 25 |
| 15.10 | 5.86 | 10 | 28.26 | 3.16 | 10 |
| 15.94 | 5.56 | 24 | 29.18 | 3.06 | 11 |
| 16.24 | 5.45 | 5 | 30.06 | 2.97 | 15 |
| 17.12 | 5.18 | 31 | 30.26 | 2.95 | 11 |
| 17.99 | 4.93 | 20 | 31.20 | 2.86 | 5 |
| 18.96 | 4.68 | 41 | 32.58 | 2.75 | 5 |
| 19.46 | 4.56 | 32 | 34.59 | 2.59 | 11 |
| 19.62 | 4.52 | 27 | 36.64 | 2.45 | 7 |
| 20.50 | 4.33 | 100 | 38.09 | 2.36 | 5 |
| 20.83 | 4.26 | 54 | 39.43 | 2.28 | 4 |

The major X-ray powder diffraction peaks are at about 11.0°, 19.0°, 20.5° and 20.8°±0.2° 2θ.

### HPLC Analysis

HPLC analysis was conducted using a mobile phase containing acetonitrile, methanol, and acetic acid at a species of gradient ratio with the flow rate of 1 ml/min. The compound was detected at a wavelength of 280 nm.

The weight percents of the tramadol free base (26.34 g) and diclofenac free acid (29.615 g) were 47% and 53% respectively in the starting mixture. The HPLC analysis of the diclofenac salt of tramadol demonstrated molar ratio of the tramadol portion and the diclofenac portion as 43.1 to 44.2, indicating that the diclofenac salt of tramadol has a 1:1 ratio of tramadol to diclofenac.

What is claimed:

1. A compound of diclofenac-tramadol salt in 1:1 ratio.

2. The compound according to claim 1, in a crystalline form.

3. The compound according to claim 2, characterized by a powder X-ray diffraction pattern, having X-ray diffraction peaks at about 11.0°, 19.0°, 20.5° and 20.8°±0.2° 2θ.

4. A method for preparing the compound according to claim 1, comprising:
dissolving a diclofenac free acid in a first solvent to form a first solution,
dissolving tramadol in a second solvent to form a second solution,
mixing the first solution and the second solution to form a mixture, and
removing the first solvent and the second solvent from the mixture to form the pharmaceutically compound.

5. The method according to claim 4, wherein said solvents are removed by natural evaporation, vacuum condensation, or drying under nitrogen.

6. The method according to claim 4, wherein the first solvent and the second solvent are selected from the group consisting of dichloromethane, ethyl acetate, methanol, ethanol, isopropyl alcohol, acetone, toluene, chloroform, dimethylformamide, dimethylacetamide, dimethylsulfoxide, methylene chloride, and acetonitrile.

7. A method for preparing the compound according to claim 1, comprising:
dissolving a diclofenac free acid and tramadol in a solvent or a solvent mixture, and
removing the solvent or the solvent mixture to form the compound.

8. The method according to claim 4, wherein said solvent or said solvent mixture is removed by natural evaporation, vacuum condensation, or drying under nitrogen.

9. The method according to claim 7, wherein said solvent or said solvent mixture is selected from the group consisting of dichloromethane, ethyl acetate, methanol, ethanol, isopropyl alcohol, acetone, toluene, chloroform, dimethylformamide, dimethylacetamide, dimethylsulfoxide, methylene chloride, acetonitrile, and a combination thereof.

10. A pharmaceutical formulation comprising the compound of claim 1 and a pharmaceutically acceptable carrier.

11. A method for treating a patient with moderate to moderately severe pain, comprising:
identifying a patient suffering from moderate to moderately severe pain with pain intensity scale of 5-9, and
administering to said patient the compound of claim 1, in an effective amount.

12. The method according to claim 11, wherein said pain is moderately severe pain with pain intensity scale of 8-9.

13. The method according to claim 11, wherein said pain is postoperative pain after Cesarean, postoperative pain after non-Cesarean surgeries, cancer pain, osteoarthritis pain, or rheumatoid arthritis pain.

14. The method according to claim 13, wherein said pain is postoperative pain after Cesarean or after non-Cesarean surgeries, and the compound is administered by intramuscular injection.

15. The method according to claim 13, wherein said pain is cancer pain and the compound is administered by oral administration.

16. The method according to claim 13, wherein said pain is osteoarthritis pain or rheumatoid arthritis pain, and the compound is administered by oral administration or topically administration.
